(19) United States
(12) Patent Application Publication
(10) Pub. No.: US 2015/0272655 A1
(43) Pub. Date: Oct. 1, 2015

(54) CONTROLLED RF ENERGY IN A MULTI-ELECTRODE CATHETER

(71) Applicant: Medtronic Ablation Frontiers, LLC, Minneapolis, MN (US)

(72) Inventors: Catherine R. CONDIE, Shoreview, MN (US); Marshall L. SHERMAN, Cardiff By The Sea, CA (US)

(73) Assignee: Medtronic Ablation Frontiers, LLC, Minneapolis, MN (US)

(21) Appl. No.: 14/227,601

(22) Filed: Mar. 27, 2014

(51) Int. Cl. A61B 18/12; A61B 18/14

(52) U.S. Cl. CPC A61B 18/1206; A61B 18/1442; A61B 2018/1253; A61B 2018/126; A61B 2018/1467; A61B 2018/00577

(57) ABSTRACT

A system and method for preventing unintended tissue damage from the delivery of unintended bipolar radiofrequency energy. The system may include a multi-electrode ablation device and an RF delivery unit. The RF delivery unit may transmit unipolar energy to the plurality of electrodes, the energy being in phase, with all electrodes delivering the same voltage and being activated at the same time to deliver no bipolar energy. Additionally or alternatively, the RF delivery unit may transmit bipolar energy to the electrodes. Here, voltage differences between each pair of adjacent electrodes may be monitored and the level of bipolar energy being delivered may be calculated. The voltage of energy delivered to at least one electrode in each adjacent electrode pair may be adjusted if the amount of delivered bipolar energy exceeds a safety threshold.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a method and system for the delivery of radiofrequency (RF) energy in a multi-electrode system. Specifically, the present invention relates to a method and system for the safe delivery of unipolar and/or bipolar RF energy in a multi-electrode system while eliminating or mitigating the delivery of unintended delivery of bipolar RF energy that may cause collateral damage to tissue.

### BACKGROUND OF THE INVENTION

Tissue ablation is a medical procedure commonly used to treat conditions such as cardiac arrhythmia, which includes atrial fibrillation. For treating cardiac arrhythmia, ablation can be performed to modify tissue, such as to stop aberrant electrical propagation and/or disrupt aberrant electrical conduction through cardiac tissue. Although non-thermal or chemical ablation may be used, tissue ablation is typically performed by delivering or removing energy from tissue, which causes the tissue to heat or cool to lethal temperatures. Other energy modalities, such as microwave energy, laser energy, and ultrasound energy may similarly cause cell damage by heating the tissue. The same procedures may be used to heat or cool tissue to non-lethal temperatures, for example, cryotreatment, cryocooling, and/or mapping procedures.

One type of frequently used thermal ablation technique is the application of radiofrequency (RF) energy to tissue. RF energy may be passed from an energy generator to one or more electrodes. When the electrodes are placed in contact with an area of target tissue, the delivery of RF energy from the one or more electrodes into the tissue may increase the temperature of the tissue to lethal temperatures.

There are two general types of RF energy delivery: unipolar and bipolar. In unipolar mode, energy travels from an electrode of a medical device (for example, an RF ablation catheter) through the target tissue. The energy may pass through the tissue to a ground or return electrode, usually located external to the patient. In bipolar mode, on the other hand, energy travels through the tissue between a first electrode and second electrode, which are usually located on the same medical device. For either energy mode, the ablation device may include more than two electrodes, and these devices may be referred to as multi-electrode devices. Unipolar RF energy delivery may cause deeper lesions than bipolar RF energy delivery. As such, unipolar RF energy delivery may be preferred when ablating thicker or tougher areas of tissue. However, well-controlled bipolar RF energy delivery may be preferred, or essential, when ablating thinner or more delicate areas of tissue or when there is concern of possible collateral damage to target or non-target tissue. However, too much bipolar energy between electrodes can cause a significant amount of local heating between the two electrodes, resulting in unintended consequences such as thermal coagulum of the blood, charring of the tissue, excessive microbubble formation, tissue overheating and steam pops, or collateral damage to target or non-target tissue.

In most currently known RF ablation systems, voltage is adjusted to change the power delivered to an electrode. When a voltage-controlled system for delivering unipolar energy to an electrode is adapted for a multi-electrode catheter or system, bipolar energy will flow between adjacent electrodes if there is a voltage difference between those electrodes. In fact, a voltage difference between adjacent electrodes frequently exists in such systems, because each individual electrode is monitored and adjusted individually based on the energy level that is required at each electrode. The resulting unintended bipolar energy can easily reach levels that are unsafe for the patient if not accounted for by the system.

It is therefore desirable to provide a method and system for ensuring the delivery of unipolar RF energy in a multi-electrode system, and for the delivery of unipolar RF energy in a multi-electrode system while preventing the unintended delivery of bipolar RF energy and/or delivering bipolar RF energy in a controlled way that prevents unintended tissue damage.

### SUMMARY OF THE INVENTION

The present invention advantageously provides a method and system for the delivery of radiofrequency (RF) energy in a multi-electrode system. Specifically, the present invention relates to a method and system for the safe delivery of unipolar and/or bipolar RF energy in a multi-electrode system while eliminating or mitigating the unintended delivery of bipolar RF energy at levels that may cause unintended damage tissue damage. In one embodiment, a system for preventing unintended tissue damage from the delivery of bipolar RF energy may include an ablation device including a plurality of electrodes and a RF energy delivery unit, the delivery unit being in electrical communication with each of the plurality of electrodes. The ablation device may further include a plurality of carrier arms, with at least one of the plurality of electrodes being located on each carrier arm. The energy delivery unit may be programmed to transmit unipolar RF energy to each of the plurality of electrodes, the transmission of RF energy to each of the plurality of electrodes being started at the same time, being in phase, and having the same voltage. The delivery unit may be further programmed to transmit RF energy having the same recurring waveform to each of the plurality of electrodes. The delivery unit may include at least one processor and a programmable logic device (PLD). The PLD may create a timing signal that causes the delivery unit to transmit RF energy having, for example, square waves. The square-wave RF energy may be filtered so that the square waveform is changed to a sinusoidal (or “sine”) waveform before the RF energy is delivered by each of the plurality of electrodes. The delivery unit may be programmed to deliver RF energy in unipolar mode only, or it may be programmed to deliver unipolar RF energy and bipolar RF energy. In the latter case, the bipolar RF energy transmitted to each electrode may include waves that are out of phase from RF energy transmitted one or more adjacent electrodes. The delivery unit may be further programmed to monitor an amount of bipolar energy delivered between each pair of adjacent electrodes. Further, monitoring the amount of bipolar energy delivered between each pair of adjacent electrodes may include monitoring a voltage difference between each pair of adjacent electrodes and/or monitoring the power delivered to each of the plurality of electrodes. The control unit may be further programmed to determine whether the amount of bipolar energy delivered by each pair of adjacent electrodes exceeds a predetermined safety threshold. For example, the predetermined safety threshold voltage may be determined before RF energy is transmitted to the plurality of electrodes. The delivery unit may be further programmed to reduce the voltage of RF energy transmitted to an electrode of a pair of adjacent electrodes that is delivering energy at a higher voltage than another of the pair of adjacent electrodes when the delivery unit determines that the amount of bipolar energy delivered between the pair of adjacent electrodes exceeds the predetermined safety threshold. For example, the voltage of RF energy transmitted to the electrode of a pair of adjacent electrodes that is delivering energy at a higher voltage than the other of the pair of adjacent electrodes is reduced such that both electrodes of the pair of adjacent electrodes each deliver RF energy having substantially the same voltage. Additionally or alternatively, the delivery unit may be further programmed to deactivate an electrode of a pair of adjacent electrodes that is delivering energy at a lower voltage than another of the pair of adjacent electrodes and/or deactivate both electrodes of the pair of electrodes, when the delivery unit determines that the amount of bipolar energy delivered between the pair of adjacent electrodes exceeds the predetermined safety threshold. Additionally or alternatively, the delivery unit may be further programmed to deliver energy to a pair of adjacent electrodes according to a duty cycle. For example, the duty cycle may include delivering RF energy at the same voltage to each electrode of the pair of adjacent electrodes.

In another embodiment, a system for preventing unintended tissue damage from the delivery of bipolar RF energy may include an ablation device including a plurality of electrodes, each of the plurality of electrodes having at least one adjacent electrode; a RF energy delivery unit in electrical communication with each of the plurality of electrodes, the RF energy delivery unit being configured to deliver RF energy including RF energy waves; and a return electrode in electrical communication with the delivery unit. The RF energy delivery unit may be programmable to: transmit unipolar RF energy to the plurality of electrodes when the RF energy transmitted to each of the plurality of electrodes is in phase, has the same voltage, and when the energy delivery unit starts the delivery of RF energy to each of the plurality of electrodes simultaneously; transmit bipolar RF energy to the plurality of electrodes when the RF waves are out of phase; monitor voltage differences between each pair of adjacent electrodes; and adjust at least one electrode in a pair of electrodes when the RF energy delivery unit determines that the voltage difference between the pair of electrodes indicates that bipolar energy is being delivered at a level that exceeds a predetermined safety threshold. Adjusting at least one electrode in a pair of electrodes may include reducing the RF energy voltage delivered by an electrode of the pair of electrodes that is delivering the higher voltage of RF energy to a voltage that is substantially the same as the voltage delivered to an electrode of the pair of electrodes that is delivering the lower voltage of RF energy. Additionally or alternatively, adjusting at least one electrode in a pair of electrodes may include deactivating an electrode of the pair of electrodes that is delivering the lower voltage of RF energy. Additionally or alternatively, adjusting at least one electrode in a pair of electrodes may include deactivating both electrodes of the pair of adjacent electrodes.

In another embodiment, a system for preventing unintended tissue damage from the delivery of bipolar RF energy may include an ablation device including a plurality of electrodes, each of the plurality of electrodes having at least one adjacent electrode and a radiofrequency energy delivery unit in electrical communication with each of the plurality of electrodes, the RF energy delivery unit being configured to deliver RF energy including RF energy waves. The RF delivery unit may be programmed to monitor voltage differences between each pair of adjacent electrodes and adjust at least one electrode in a pair of electrodes when the radiofrequency energy delivery unit determines that the voltage difference between the pair of electrodes indicates that bipolar energy is being delivered at a level that exceeds a predetermined safety threshold. Adjusting at least one electrode in a pair of electrodes may include at least one of: reducing the voltage of RF energy transmitted to an electrode of a pair of electrodes that is delivering energy at a higher voltage than another of the pair of electrodes; and deactivating an electrode of a pair of electrodes that is delivering energy at a lower voltage than another of the pair of electrodes. The RF energy delivery unit may further be programmed to monitor power differences between each pair of adjacent electrodes and adjust at least one electrode in a pair of electrodes when the RF energy delivery unit determines that the power difference between the pair of electrodes indicates that bipolar energy is being delivered at a level that exceeds a predetermined safety threshold.

In one embodiment, a method for preventing unintended tissue damage may include transmitting unipolar RF energy to a plurality of electrodes of a multi-electrode medical device, the RF energy being transmitted coherently to each of the plurality of electrodes, the RF transmitted to each of the plurality of electrodes being in phase with the RF energy delivered to the other of the plurality of electrodes and the RF energy transmitted to each of the plurality of electrodes having the same voltage. The method may further include transmitting RF energy to each of the plurality of electrodes according to a duty cycle. Further, each of the plurality of electrodes may have at least one adjacent electrode to create a pair of adjacent electrodes, the plurality of electrodes including a plurality of adjacent pairs of electrodes. The method may further comprise: transmitting bipolar RF energy between at least one pair of adjacent electrodes when RF energy delivered to a first electrode of the pair of adjacent electrodes is out of phase with or at a different voltage than RF energy delivered to a second electrode of the pair of adjacent electrodes; monitoring voltage differences between the at least one pair of adjacent electrodes; and adjusting RF energy delivered to at least one electrode in the at least one pair of adjacent electrodes when the RF energy delivery unit determines that the voltage difference between the electrodes of the at least one pair of adjacent electrodes indicates that bipolar energy is being delivered at a level that exceeds a predetermined safety threshold. Adjusting at least one electrode in the at least one pair of adjacent electrodes may include at least one of: reducing the RF energy voltage transmitted to an electrode of the at least one pair of adjacent electrodes that is delivering the higher voltage of RF energy such that both electrodes in the at least one pair of adjacent electrodes deliver substantially the same voltage; and deactivating an electrode of the at least one pair of adjacent electrodes that is delivering the lower voltage of RF energy.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows an exemplary multi-electrode RF ablation system;

FIG. 2 shows an exemplary multi-electrode RF device;

FIG. 3A shows a schematic view of unipolar in-phase energy delivery in a multi-electrode RF ablation system;

FIG. 3B shows a schematic view of out-of-phase energy delivery in a multi-electrode

RF ablation system;

FIG. 4A shows a method of delivering unipolar energy in a multi-electrode system; and

FIG. 4B shows a method of delivering controlled amounts of bipolar energy in a multi-electrode system.

### DETAILED DESCRIPTION OF THE INVENTION

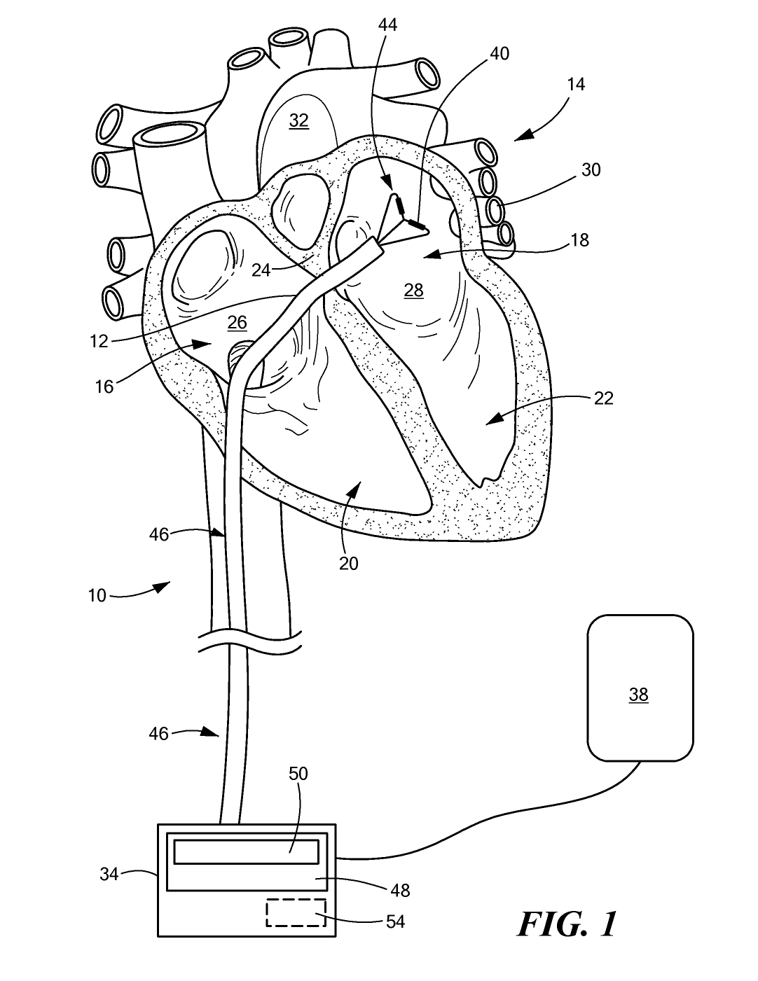

Referring now to FIG. 1, an exemplary multi-electrode RF ablation system is shown. The system 10 may be used to treat endocardial surfaces, and the device 12 in FIG. 1 is shown positioned within a heart. However, it will be understood that the system 10 may be used to treat other areas, including epicardial tissue, esophageal tissue, dermal tissue, and any other tissue that is treated with radiofrequency (RF) energy. The cross-sectional view of the heart 14 shows the major structures, including the right atrium 16, the left atrium 18, the right ventricle 20, and the left ventricle 22. The atrial septum 24 separates the right 16 and left 18 atria. In a patient suffering from atrial fibrillation, for example, aberrant electrical conduction may be found in tissue of the atrial walls 26, 28, as well as of the pulmonary veins 30 and the pulmonary arteries 32. Ablation of these areas, which may be referred to as arrhythmogenic foci, drivers, or rotors, may be an effective treatment for atrial fibrillation. Unipolar RF energy delivery may be preferred when ablating thicker or tougher areas of tissue, but well-controlled bipolar RF energy delivery may be preferred, or essential, when ablating thinner or more delicate areas of tissue. However, too much bipolar energy between electrodes can cause a significant amount of local heating between the two electrodes, resulting in unintended consequences such as thermal coagulum of the blood, charring of the tissue, excessive microbubble formation, tissue overheating and steam pops, or collateral damage to target or non-target tissue. Therefore, a user may prefer to either delivery unipolar energy only. If the user wants to use bipolar RF energy either in additional to or instead of unipolar RF energy, however, the application of the bipolar RF energy must be carefully controlled so as not to cause collateral damage.

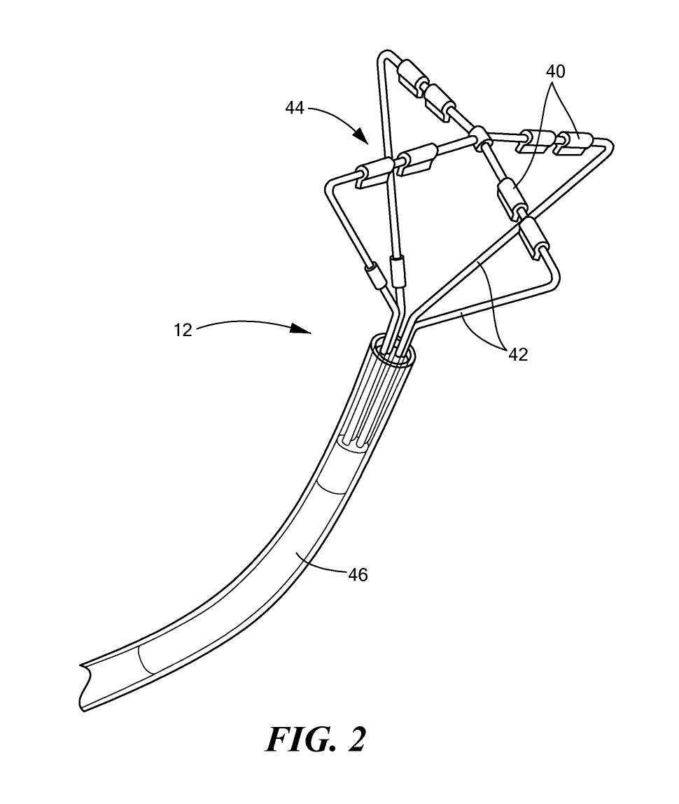

Referring to FIGS. 1 and 2, the system 10 may generally include a device 12, an RF delivery unit 34, and a return electrode 38. The device 12 may be a multi-electrode device 12. For example, a plurality of electrodes 40 may be grouped as an array, with one or more electrodes 40 each borne on a carrier arm 42. A non-limiting example of this configuration is shown in FIG. 2. The carrier arms 42 may together be referred to as a carrier assembly 44, which may be coupled to or disposed within the device elongate body 46. For example, the carrier assembly 44 may be slidably disposed within the elongate body 46. In this case, the carrier assembly 44 may have a first collapsed configuration that enables the carrier assembly 44 to be completely or substantially disposed within the elongate body during delivery of the device 12 to the target treatment site. Once at the target treatment site, the carrier assembly 44 may be advanced distally from the elongate body 46, at which point the carrier assembly 44 transitions from the first collapsed configuration to an expanded configuration, such as that shown in FIG. 2. The carrier assembly 44 may be deformable such that pressing the carrier assembly 44 into, for example, an atrial wall may cause one or more electrodes 40 to make contact with the tissue to be treated. The electrodes 40 may be fin-shaped (as shown in FIG. 2) or may be disposed about at least a portion of the outer surface of at least a portion of each carrier arm 42. Further, the electrodes 40 may protrude from the surface of the carrier arms 42 (for example, as shown in FIG. 2) or may be flush or substantially flush with the surface of the carrier arms. Each of the electrodes 40 may be in electrical communication with the RF delivery unit 34, which is also in electrical communication with the return electrode 38. Although not shown, the carrier assembly 44 may also include one or more sensors for communicating data such as temperature, pressure, electrical impedance, and the like to the control unit 36 for the automatic or manual adjustment of system parameters.

Referring again to FIG. 1, the RF delivery unit 34 may be configured to deliver RF energy in unipolar, bipolar, or combination unipolar-bipolar energy delivery modes. For example, the RF delivery unit 34 may be configured to deliver duty-cycled phased RF energy. Further, the RF delivery unit 34 may be a multichannel delivery unit, capable of independently and selectively delivering RF energy to each electrode 40. The RF delivery unit 34 may also be configured to provide electrical mapping of tissue that is contacted by one or more electrodes 40 of the carrier assembly 44. Likewise, the electrodes 40 may also be configured to be mapping electrodes and/or additional electrodes can be included on the carrier assembly 44 to provide mapping functionality. Energy provided by the RF delivery unit 34 may be sufficient to heat tissue to a temperature of approximately 60° C. or more. Further, the RF delivery unit 34 may serve as a control unit and may include a user interface 48 by which the user may select the energy delivery mode, monitor energy delivery parameters, adjust or stop energy delivery, and/or select one or more electrodes to which to deliver energy. For example, the user interface 48 may include a mouse, joystick, one or more displays 50, buttons, knobs, touchpads, touchscreens, or other input means. Although the system 10 and energy delivery may be completely automated, the user may control the form of the RF waves, on/off status of individual electrodes, and/or delivery voltage through the user interface 48.

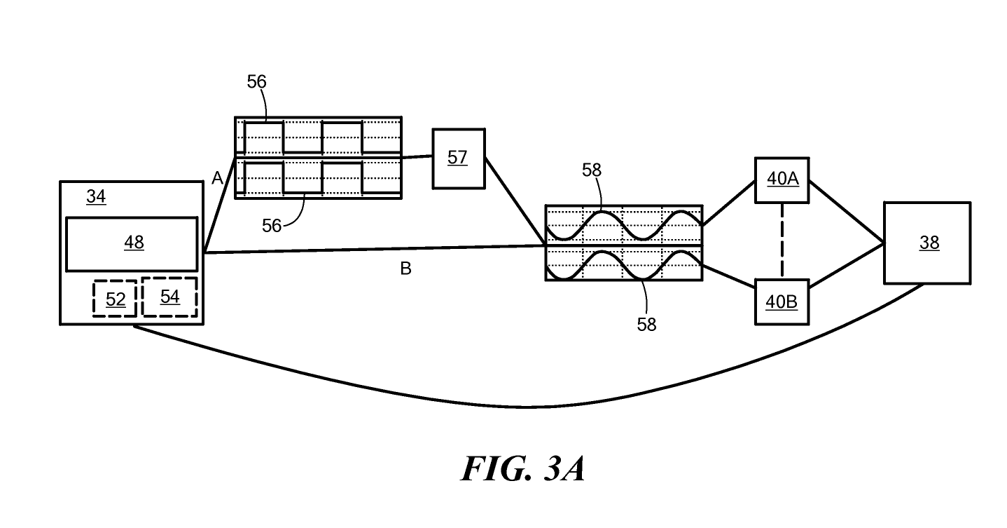

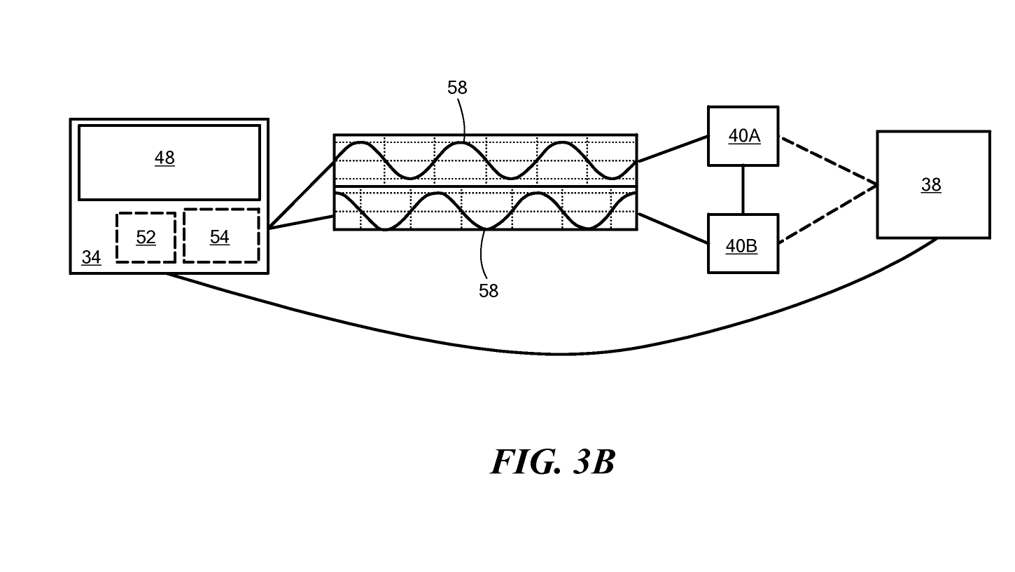

Referring now to FIGS. 3A and 3B, schematic views of a multi-electrode RF ablation system delivering unipolar and/or controlled bipolar energy are shown. The RF delivery unit 34 may further include a programmable logic device 52 (PLD) and one or more processors 54. The PLD 52 may be, for example, a binary logic device. The RF delivery unit 34 may be configured and programmable to prevent or limit unintended bipolar energy in two ways, either together or individually. First, RF energy delivered by two or more electrodes 40 may be delivered in such a way as to ensure operation of the device 12 in unipolar mode. For example, RF energy delivered by the electrodes 40 may be delivered such that energy transmission to each electrode may be started and stopped at the same time, may be in-phase, may be delivered at the same voltage, and the energy delivered by each electrode may have the same waveform (as shown in FIG. 3A). For simplicity, this may be referred to as “matched RF delivery.” This method of matched delivery may be used in voltage-controlled multi-electrode systems and multi-electrode systems that are not voltage-controlled. As a non-limiting example of a method for accomplishing this matched RF delivery, the PLD 52 may be used to create timing signals or timing circuit that causes the generation of RF energy waves having a square shape. The square shape of the waves may allow for precise timing and synchronization of energy transmission from the delivery unit 34 (that is, the transmission of energy to each electrode 40 may be started at stopped at the same time). However, it will be appreciated that any recurring waveform shape may be used, as long as energy transmitted to all electrodes has the same shape. The square waves 56 (or other waves having a recurring waveform) may then be filtered 57 so as to be transformed into sinusoidal waves 58 (as shown as path A in FIG. 3A) before being delivered by the electrodes 40 to the tissue. Alternatively, energy transmitted from the delivery unit 34 may be sinusoidal waves 58, which do not need to be filtered for shape, although they may be filtered for other characteristics (as shown as path B in FIG. 3A). As is shown by the solid lines from electrodes 40A and 40B to the return electrode 38, no bipolar RF energy will be delivered between the electrodes 40A, 40B when energy transmitted to the electrodes 40A, 40B is started and stopped at the same time, is in phase, has the same waveform, and has the same voltage.

As discussed, RF energy may be delivered from each of the electrodes 40 as sinusoidal waves 58. Although a user may intend to deliver only unipolar RF energy from the electrodes 40, unintended phase shifts may occur between electrodes 40 (for example, as shown in FIG. 3B). Alternatively, the user may intend to deliver controlled amounts of bipolar energy. If any phase difference between electrodes exists, bipolar energy will be delivered, and the amount of this bipolar energy delivered may be significant. For example, a 60° phase shift may result in a 100V difference between the two electrodes, meaning that 100V of bipolar energy is being delivered to the tissue. Thus, in an additional or alternative method of preventing or limiting unintended bipolar energy, the RF delivery unit 34 may be programmed to monitor the voltage of each activated electrode during energy delivery to control the delivery of bipolar energy. The RF delivery unit 34 may also be programmed to monitor delivered power as well as delivered voltage, and to likewise adjust energy transmitted to the electrodes 40 accordingly. For example, the one or more processors 54 of the RF delivery unit 34 may execute one or more algorithms to monitor electrode voltages. If the user wants to deliver bipolar RF energy, the user and/or the control unit 34 may carefully monitor the phase shift and resulting bipolar RF energy being delivered between adjacent electrodes 40. For example, the amount of bipolar RF energy delivered between adjacent electrodes 40 when a phase shift is present may be determined by the following equation:

Bipolar RF voltage=(sin(degrees phase shift/2)'2)×unipolar RF voltage

As a non-limiting example for illustration only, the phase shift may be 180° and the unipolar RF voltage may be 100V. Using the above equation, the 180° phase shift will result in a 200V difference between the adjacent electrodes 40, meaning that 200V of bipolar energy is being delivered.

If a phase shift, either intended or unintended, generates an amount of bipolar RF energy that is greater than desired by the user (for example, because the amount of RF energy delivered would cause collateral damage to target or non-target tissue), the control unit 34 may be programmed or programmable to transmit energy to adjacent electrodes according to a duty cycle and/or to automatically deactivate one of an adjacent pair of electrodes 40.

Currently known systems adjust the voltage of energy being transmitted to electrodes in order to control the ablative effect of a treatment on target tissue. That is, voltage delivered to an electrode may be adjusted to produce a desired electrode temperature. However, because this adjustment is made at each electrode based on monitoring that electrode in isolation from adjacent electrodes, such adjustment can result in the unintended delivery of bipolar RF energy. For example, in a currently known multi-electrode unipolar RF energy delivery system, the voltage of each electrode may be monitored to determine the temperature being delivered to the tissue. An electrode may have a first surface that is in contact with tissue and a second surface that is in contact with flowing blood rather than tissue. The flowing blood helps cool the second surface of the electrode, which causes the temperature of the first side of the electrode to increase. As the first side of the electrode ablates the tissue, the electrode may sink into the tissue (referred to as becoming buried within the tissue). Although this is a desired effect, it may reduce or eliminate contact between the second side of the electrode and the flowing blood. As a result, the electrode may need only a fraction of the original power to effectively ablate tissue, and delivering the original amount of energy may result in tissue charring. In order to reduce the amount of energy being delivered, the system may reduce the voltage of the buried electrode. Although this may be effective to reduce the temperature of that electrode, it may also result in the unintended delivery of bipolar energy between that electrode and an adjacent electrode, often in an amount that exceeds a predetermined safety threshold. For example, 100V of energy may be delivered to each of two adjacent electrodes. If one electrode becomes buried in the tissue, that electrode may need only 10V to produce the correct electrode temperature. If the system reduces the voltage on that electrode to 10V, there is now a 90V difference between the two adjacent electrodes. That is, 90V of unintended bipolar energy is being delivered between the adjacent electrodes.

Unlike currently known systems, the present system either does not adjust voltage of individual electrodes to control electrode temperature or does so only after determining the resulting bipolar effect between adjacent electrodes. In the first case, the voltage of energy delivered to all electrodes may be the same and constant, but each electrode 40 may be operated according to a duty cycle in which the electrode 40 is activated for a certain amount of time and deactivated for a certain amount of time. For example, if 100 watts of RF energy is being delivered but only 10 watts is required to produce the desired electrode temperature, that electrode 40 may be activated for 10% of a given period of time and deactivated for 90% of that duration of time. The delivery unit 34 may be programmed to execute an algorithm that determines the correct duty cycle based on, for example, transmitted voltage, duration treatment time, electrode temperature, or other factors. Additionally, the delivery unit 34 may be programmed to create a duty cycle for one or more electrodes 40 as needed, based at least in part on, for example, temperature data received from one or more temperature sensors on one or more electrodes 40. When an electrode 40 is deactivated (rather than reduced to 0V), it may be referred to as being in a high-impedance state. Thus, when an electrode is in a high-impedance state, no bipolar energy is possible between the deactivated or high-impedance electrode and an adjacent electrode, even though the electrode is being maintained at a desired temperature. Further, even if two adjacent electrodes are delivering energy at the same time (for example, each electrode is at an activation stage of its duty cycle), no bipolar energy will be delivered between the electrodes because the delivery unit 34 is transmitting energy at the same voltage to all electrodes. For this reason, the energy pathway between electrodes 40A and 40B in FIG. 3B is shown as a dashed line, because bipolar energy may change to unipolar energy if the voltages at each electrode 40A, 40B are the same or if one of an adjacent pair of electrodes is deactivated.

In the second case, the delivery unit 34 may monitor the voltage of energy delivered by each electrode and the amount of bipolar energy delivered between adjacent electrodes 40. The determined amount of bipolar energy may be compared to a predetermined safety threshold to determine whether the amount of bipolar energy is a safe amount or whether it is likely to cause collateral damage, such as tissue charring and/or unintended damage to non-target tissue. For example, the delivery unit 34 may make the comparison based on voltage differences between adjacent electrodes 40 and/or bipolar power delivered, which may be calculated as the product of the current and the voltage. Based on this comparison, the delivery unit 34 may reduce the voltage of the higher of the two electrodes 40 if the delivered bipolar energy is above a safe level. That is, if a first electrode 40A is delivering RF energy at a greater voltage than an adjacent electrode 40B, the voltage of energy delivered by electrode 40A may be reduced in order to reduce or eliminate the amount of bipolar RF energy being delivered to the target tissue. As a non-limiting embodiment, if electrode 40A is delivering RF energy at 40V and electrode 40B is delivering RF energy at 30V, the voltage delivered by electrode 40A may be reduced to 30V. Additionally or alternatively, the delivery unit 34 may be programmed or programmable to deactivate the lower of the two electrodes 40 if the delivered bipolar energy is above a safe level. That is, if a first electrode 40A is delivering RF energy at a greater voltage than an adjacent electrode 40B, electrode 40B may be deactivated (that is, transitioned to a high-impedance state), thereby preventing the delivery of bipolar RF energy between the electrodes 40A, 40B. As a non-limiting example, this method of preventing the delivery of unintended bipolar energy may be useful in existing voltage-controlled systems.

In these methods, bipolar energy may be delivered in a carefully controlled way, so that the user may apply bipolar energy before, during, or instead of unipolar energy while mitigating or eliminating the chance of unintended tissue damage. The threshold above with bipolar energy is not being delivered at a safe level may be determined empirically and/or based on individual patient characteristics. Further, this threshold may be determined before the delivery of ablation energy begins, and this predetermined threshold may be programmed into the RF delivery unit 34 through, for example, the user interface 48.

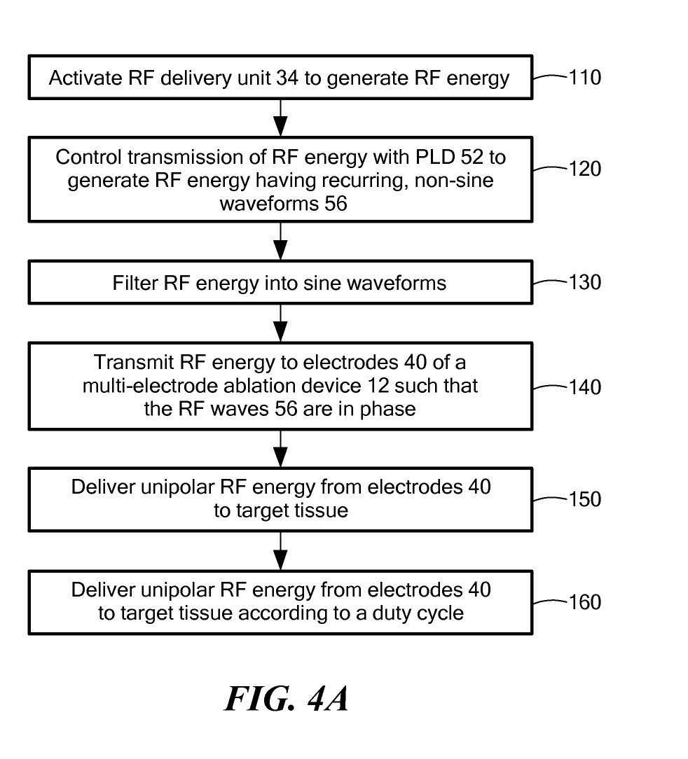

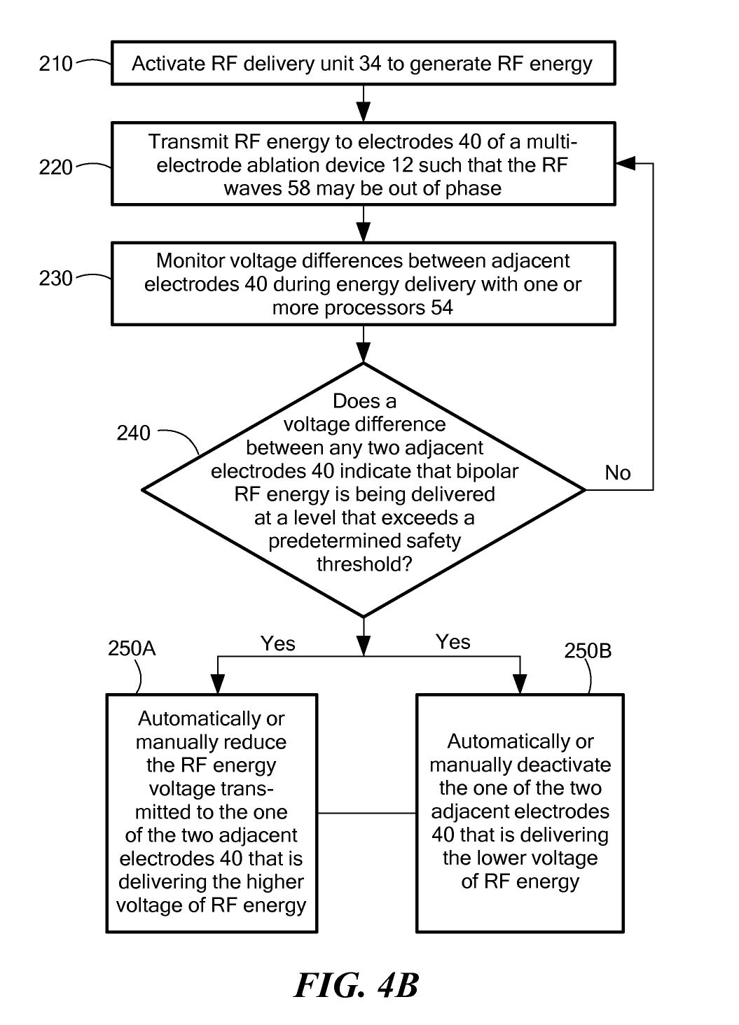

Referring now to FIG. 4A, a method of delivering unipolar energy in a multi-electrode RF ablation system is shown. In the first step 110, the RF delivery unit 34 may be activated (for example, turned on and instructed to transmit RF energy to the electrodes 40). As energy is delivered from the RF delivery unit 34, the PLD 52 may instruct (that is, create timing signals that cause) the delivery unit 34 to produce and transmit RF energy having a recurring, non-sinusoidal waveform (for example, a square waveform) in the second step 120. In the third step 130, the RF energy may then be filtered by one or more processors 54 or other components of the RF delivery unit 34 into sinusoidal waves. However, it will be understood that sinusoidal-wave RF energy may be transmitted from the delivery unit 34 to the electrodes without performing the second 120 and third steps 130. In the fourth step 140, the RF energy is transmitted to the electrodes 40 of a multi-electrode ablation device 12 such that the RF energy waves 56 are in phase (that is, no phase shift exists between RF energy delivered by adjacent electrodes 40). Also, energy may be delivered coherently to the plurality of electrodes 40, meaning that energy delivery to each electrode 40 may be started at the same time as the other electrodes 40 and may be stopped at the same time as the other electrodes 40. If the fifth step 150, energy may be delivered from the electrodes 40 to the target tissue. Further, all electrodes may deliver energy having the same voltage. Optionally, in the sixth step 160, energy may be transmitted to and delivered from each electrode according to a duty cycle established automatically by the delivery unit 34 and/or manually by the user. Using a duty-cycled energy delivery may ensure that each electrode applies a correct amount of ablation energy to the target tissue without adjusting the voltage of energy delivered. Because each electrode receives and delivers energy having the same voltage, there will be no bipolar energy delivered between adjacent electrodes, even if both electrodes are active at the same time. In this manner, unipolar energy may be safely delivered to a target area of tissue without the risk of delivering unintended bipolar energy.

Referring now to FIG. 4B, a method of delivering controlled bipolar energy in a multi-electrode RF ablation system is shown. In the first step 210, the RF delivery unit 34 may be activated (for example, turned on and instructed to transmit RF energy to the electrodes 40). The RF energy delivered to the electrodes 40, in the second step 220, may include sinusoidal waves 58, and there may be some phase shift between waves of RF energy delivered between adjacent electrodes 40. Therefore, some bipolar RF energy may be delivered to the target tissue. Although the phase shift may occur unintentionally, it will be understood that a user and/or the delivery unit 34 may intentionally create the phase shift in order to apply bipolar energy. To prevent unintended tissue damage, the system 10 may monitor the transmission of this bipolar energy in the third step 230. Based on the differences in voltage delivered to adjacent electrodes 40, the system 10 may determine whether a voltage difference between any two adjacent electrodes 40 indicates that bipolar RF energy is being delivered to target tissue at a level that exceeds the predetermined safety threshold in the fourth step 240. If not, energy may continue to be transmitted to all electrodes 40 without modification.

If bipolar energy delivered does exceed the safety threshold, however, the user and/or system 10 may perform either or both of the steps in FIG. 4B identified by reference numbers 250A and 250B. In the step identified as 250A, the one or more processors 54 of the RF delivery unit 34 may execute one or more algorithms to monitor electrode voltages. As each electrode 40 delivers RF energy, the voltage on adjacent electrodes 40 may be varied. The RF delivery unit 34 may determine the amount of bipolar energy being delivered by monitoring the voltage differences between adjacent electrodes 40. The RF delivery unit 34 may monitor this bipolar energy and automatically reduce the voltage transmitted to and delivered by the higher of the two electrodes 40 if the delivered bipolar energy is above a safe level. For example, the voltage of energy transmitted to and delivered by one electrode may be reduced until it is the same or substantially the same voltage transmitted to and delivered by the second electrode. The adjustment may be made automatically by the system 10 or the system 10 may communicate one or more system parameters to the user, who may then manually adjust the voltage of energy delivered to one or more electrodes 40 and/or override automated system operation. In this manner, the delivery of bipolar energy to the target tissue may be controlled so that delivered energy does not exceed a predetermined safety threshold.

Continuing to refer to FIG. 4B, in the step identified as 250B, the RF delivery unit 34 may be programmed to deactivate one or more electrodes 40, or the user may manually deactivate one or more electrodes 40, to terminate RF energy delivery between adjacent electrodes. For example, the RF delivery unit 34 may be programmed to deactivate an electrode 40 (that is, transition the electrode 40 to a high-impedance state) that the RF delivery unit 34 determines is delivering a lower energy level (or greater amount of energy) than an adjacent electrode 40. In doing this, the delivery unit 34 may simultaneously or sequentially compare sets of two adjacent electrodes so that each electrode is evaluated in comparison to each adjacent electrode. In these methods, bipolar energy may be delivered in a carefully controlled way, so that the user may apply bipolar energy before, during, or instead of unipolar energy while mitigating or eliminating the chance of unintended tissue damage.

It will be understood that the methods and systems disclosed herein may be used in any multi-electrode RF ablation system, including voltage-controlled systems. Thus, the methods and systems of the present invention may be implemented in an existing RF ablation system in order to prevent the delivery of bipolar RF energy and/or to control the delivery of bipolar RF energy so that bipolar energy is delivered at levels that do not exceed a predetermined safety threshold. Further, both the method of FIG. 4A and the method of FIG. 4B may be used during the same medical procedure. In a non-limiting example, RF energy may be delivered to target tissue first in unipolar mode (such as shown in FIG. 4A) and then in controlled bipolar mode (such as shown in FIG. 4B). It will also be understood that additional steps may be performed in each method even if not shown in the figures. For example, multiple monitoring steps may be performed at various stages, or the system 10 may be continuously monitored, throughout the duration of the medical procedure to enhance patient safety. Additionally, the RF delivery unit 34 may deliver one or more visible or audible alerts to communicate system and/or operational parameters to the user, and the like.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A system for preventing unintended tissue damage from the delivery of bipolar radiofrequency energy, the system comprising:
an ablation device including a plurality of electrodes; and
a radiofrequency energy delivery unit, the delivery unit being in electrical communication with each of the plurality of electrodes;
the energy delivery unit being programmed to transmit unipolar radiofrequency energy to each of the plurality of electrodes, the transmission of radiofrequency energy to each of the plurality of electrodes being started at the same time, being in phase, and having the same voltage.

2. The system of claim 1, wherein the energy delivery unit is further programmed to transmit radiofrequency energy having a same recurring waveform to each of the plurality of electrodes.

3. The system of claim 1, wherein the delivery unit includes at least one processor and a programmable logic device.

4. The system of claim 3, wherein the programmable logic device creates a timing signal that causes the delivery unit to transmit radiofrequency energy having square waves.

5. The system of claim 4, wherein the radiofrequency energy having square waves is filtered such that the radiofrequency energy has a sinusoidal waveform, the sinusoidal waveform radiofrequency energy being delivered by each of the plurality of electrodes.

6. The system of claim 1 wherein the delivery unit is programmed to delivery radiofrequency energy in unipolar mode only.

7. The system of claim 1, wherein the energy delivery unit is further programmed to transmit bipolar radiofrequency energy.

8. The system of claim 7, wherein the bipolar radiofrequency energy transmitted to each electrode includes waves that are out of phase from radiofrequency energy transmitted one or more adjacent electrodes.

9. The system of claim 7, wherein the delivery unit is further programmed to monitor an amount of bipolar energy delivered between each pair of adjacent electrodes.

10. The system of claim 9, wherein monitoring the amount of bipolar energy delivered between each pair of adjacent electrodes includes at least one of monitoring a voltage difference between each pair of adjacent electrodes and the power delivered to each of the plurality of electrodes.

11. The system of claim 10, wherein the control unit is further programmed to determine whether the amount of bipolar energy delivered by each pair of adjacent electrodes exceeds a predetermined safety threshold.

12. The system of claim 11, wherein the predetermined safety threshold voltage is determined before radiofrequency energy is transmitted to the plurality of electrodes.

13. The system of claim 11, wherein the delivery unit is further programmed to reduce the voltage of radiofrequency energy transmitted to an electrode of a pair of adjacent electrodes that is delivering energy at a higher voltage than another of the pair of adjacent electrodes when the delivery unit determines that the amount of bipolar energy delivered between the pair of adjacent electrodes exceeds the predetermined safety threshold.

14. The system of claim 13, wherein the voltage of radiofrequency energy transmitted to the electrode of a pair of adjacent electrodes that is delivering energy at a higher voltage than the other of the pair of adjacent electrodes is reduced such that both electrodes of the pair of adjacent electrodes each deliver radiofrequency energy having substantially the same voltage.

15. The system of claim 11, wherein the delivery unit is further programmed to at least one of deactivate an electrode of a pair of adjacent electrodes that is delivering energy at a lower voltage than another of the pair of adjacent electrodes and deactivating both electrodes of the pair of adjacent electrodes, when the delivery unit determines that the amount of bipolar energy delivered between the pair of adjacent electrodes exceeds the predetermined safety threshold.

16. The system of claim 11, wherein the delivery unit is further programmed to deliver energy to a pair of adjacent electrodes according to a duty cycle.

17. The system of claim 16, wherein the duty cycle includes delivering radiofrequency energy at the same voltage to each electrode of the pair of adjacent electrodes.

18. The system of claim 1, wherein the ablation device further includes a plurality of carrier arms, at least one of the plurality of electrodes being located on each carrier arm.

19. A system for preventing unintended tissue damage from the delivery of bipolar radiofrequency energy, the system comprising:
an ablation device including a plurality of electrodes, each of the plurality of electrodes having at least one adjacent electrode;
a radiofrequency energy delivery unit in electrical communication with each of the plurality of electrodes, the radiofrequency energy delivery unit being configured to deliver radiofrequency energy including radiofrequency energy waves; and
a return electrode in electrical communication with the delivery unit, the radiofrequency energy delivery unit being programmable to:
transmit unipolar radiofrequency energy to the plurality of electrodes when the radiofrequency energy transmitted to each of the plurality of electrodes is in phase, has the same voltage, and when the energy delivery unit starts the delivery of radiofrequency energy to each of the plurality of electrodes simultaneously;
transmit bipolar radiofrequency energy to the plurality of electrodes when the radiofrequency waves are out of phase;
monitor voltage differences between each pair of adjacent electrodes; and
adjust at least one electrode in a pair of electrodes when the radiofrequency energy delivery unit determines that the voltage difference between the pair of electrodes indicates that bipolar energy is being delivered at a level that exceeds a predetermined safety threshold.

20. The system of claim 19, wherein adjusting at least one electrode in a pair of electrodes includes reducing the radiofrequency energy voltage delivered by an electrode of the pair of electrodes that is delivering the higher voltage of radiofrequency energy to a voltage that is substantially the same as the voltage delivered to an electrode of the pair of electrodes that is delivering the lower voltage of radiofrequency energy.

21. The system of claim 19, wherein adjusting at least one electrode in a pair of electrodes includes deactivating an electrode of the pair of electrodes that is delivering the lower voltage of radiofrequency energy.

22. The system of claim 19, wherein adjusting at least one electrode in a pair of electrodes may include deactivating both electrodes of the pair of adjacent electrodes.

23. A system for preventing unintended tissue damage from the delivery of bipolar radiofrequency energy, the system comprising:
an ablation device including a plurality of electrodes, each of the plurality of electrodes having at least one adjacent electrode; and
a radiofrequency energy delivery unit in electrical communication with each of the plurality of electrodes, the radiofrequency energy delivery unit being configured to deliver radiofrequency energy including radiofrequency energy waves, the radiofrequency energy delivery unit being programmed to:
monitor voltage differences between each pair of adjacent electrodes; and
adjust at least one electrode in a pair of electrodes when the radiofrequency energy delivery unit determines that the voltage difference between the pair of electrodes indicates that bipolar energy is being delivered at a level that exceeds a predetermined safety threshold.

24. The system of claim 23, wherein adjusting at least one electrode in a pair of electrodes includes at least one of:
reducing the voltage of radiofrequency energy transmitted to an electrode of a pair of electrodes that is delivering energy at a higher voltage than another of the pair of electrodes; and
deactivating an electrode of a pair of electrodes that is delivering energy at a lower voltage than another of the pair of electrodes.

25. The system of claim 23, wherein the radiofrequency energy delivery unit is further programmed to monitor power differences between each pair of adjacent electrodes and adjust at least one electrode in a pair of electrodes when the radiofrequency energy delivery unit determines that the power difference between the pair of electrodes indicates that bipolar energy is being delivered at a level that exceeds a predetermined safety threshold.

26. A method for preventing unintended tissue damage, the method comprising:
transmitting unipolar radiofrequency energy to a plurality of electrodes of a multi-electrode medical device, the radiofrequency energy being transmitted coherently to each of the plurality of electrodes, the radiofrequency transmitted to each of the plurality of electrodes being in phase with the radiofrequency energy delivered to the other of the plurality of electrodes, and the radiofrequency energy transmitted to each of the plurality of electrodes having the same voltage.

27. The method of claim 26, further comprising transmitting radiofrequency energy to each of the plurality of electrodes according to a duty cycle.

28. The method of claim 27, wherein each of the plurality of electrodes has at least one adjacent electrode to create a pair of adjacent electrodes, the plurality of electrodes including a plurality of adjacent pairs of electrodes, the method further comprising:
transmitting bipolar radiofrequency energy between at least one pair of adjacent electrodes when radiofrequency energy delivered to a first electrode of the pair of adjacent electrodes is out of phase with or at a different voltage than radiofrequency energy delivered to a second electrode of the pair of adjacent electrodes;
monitoring voltage differences between the at least one pair of adjacent electrodes; and
adjusting radiofrequency energy delivered to at least one electrode in the at least one pair of adjacent electrodes when the radiofrequency energy delivery unit determines that the voltage difference between the electrodes of the at least one pair of adjacent electrodes indicates that bipolar energy is being delivered at a level that exceeds a predetermined safety threshold.

29. The method of claim 28, wherein adjusting at least one electrode in the at least one pair of adjacent electrodes includes at least one of:
reducing the radiofrequency energy voltage transmitted to an electrode of the at least one pair of adjacent electrodes that is delivering the higher voltage of radiofrequency energy such that both electrodes in the at least one pair of adjacent electrodes deliver substantially the same voltage; and
deactivating an electrode of the at least one pair of adjacent electrodes that is delivering the lower voltage of radiofrequency energy.
